SEQUENCE LISTING (1 sequence)

SEQ ID NO: 1 - protein, 748 aa, Homo sapiens
  Polypeptide
AAQPARRARRTKLFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMQHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSADIQHSGGRSSLEGPRFEQKLISEEDLNMHTGHHHHHH

(19) United States
(12) Patent Application Publication
(10) Pub. No.: US 2009/0226367 A1
(43) Pub. Date: Sep. 10, 2009

(54) COMPOSITIONS COMPRISING AN ANTI-CA125 ANTIBODY AND A CYTOTOXIC COMPOUND AND THEIR USE FOR THE TREATMENT OF CANCER

(75) Inventor: Earl F. Albone, Plymouth Meeting, PA (US)

Correspondence Address: SEED INTELLECTUAL PROPERTY LAW GROUP PLLC, 701 FIFTH AVE, SUITE 5400, SEATTLE, WA, 98104, US

(73) Assignee: Euro-Celtique S.A., Luxembourg (LU)

(21) Appl. No.: 11/908,355

(22) PCT Filed: Mar. 8, 2006
(86) PCT No.: PCT/US2006/008659

§ 371 Date: Jun. 26, 2008

Related U.S. Application Data
(60) Provisional application No. 60/661,309, filed on Mar. 11, 2005.

(51) Int. Cl. A61K 51/00; A61K 39/395

(52) U.S. Cl. 424/1.49; 424/174.1; 424/178.1; 424/156.1; 424/139.1

(57) ABSTRACT

The present invention provides combination therapy methods, formulations and kits useful for managing or treating a CA 125-related disorder or symptom thereof. In one aspect, the present invention provides methods for managing or treating a cell proliferative disorder, such as a cancer, for example, ovarian cancer, comprising administering a combination of (a) a sensitizer, such as, for example, paclitaxel, and (b) an antibody that preferentially binds cell-associated CA 125 polypeptides relative to shed CA 125 polypeptides, hi some embodiments of the methods provided, the antibody is conjugated to a cytotoxic agent such as a radiolabel. In another aspect, the present invention provides pharmaceutical compositions comprising (a) a sensitizer; (b) an antibody that preferentially binds cell-associated CA 125 polypeptides relative to shed CA 125 polypeptides; and, (c) a pharmaceutically acceptable excipient, diluent, or carrier.

### 1. REFERENCE TO PRIOR APPLICATION

This application claims the benefit of U.S. Provisional Application No. 60/661,309, filed Mar. 11, 2005.

### 2. FIELD OF THE INVENTION

The present invention relates to methods, formulations and kits for the management or treatment of a symptom of a CA 125-related disorder, preferably a cell proliferative disorder such as a cancer such as, for example, an ovarian cancer. For example, methods of managing a cell proliferative disorder are provided comprising administering to a subject a combination of a sensitizer and an antibody, wherein the antibody preferentially binds a cell-associated CA 125 polypeptide relative to shed CA 125 polypeptide. In certain embodiments of the methods provided, the sensitizer is paclitaxel and the antibody is a monoclonal antibody conjugated to a radionuclide or another cytotoxic agent.

### 3. BACKGROUND OF THE INVENTION

Ovarian cancer is the fifth most common cause of cancer-related mortality among female cancer patients in the United States. Due to its tendency to spread into the peritoneal cavity, the bowel, and the bladder, and the relatively asymptomatic progression of ovarian cancer, the majority of cancer patients present with advanced disease and have a poor long-term prognosis.

CA 125 is a cell surface mucin glycoprotein that is overexpressed in a majority of ovarian cancers of epithelial origin. This molecule is shed from the surface of tumor cells and the serum levels of the shed protein are used clinically to monitor patients for the recurrence of disease following primary surgical debulking and chemotherapy. In vitro studies suggest that taxanes increase the expression of CA 125 in ovarian carcinoma cell lines (see Marth et al., Cancer Res. 57:3818-3822 (1997)). In addition to antibodies for monitoring the presence of CA 125, U.S. Pat. Nos. 5,858,361 and 6,241,985 describe anti-idiotypic anti-CA 125 antibodies as therapeutic agents. WO 2004/035537 (PCT/US2003/032945) describes antibodies that demonstrate a preference for binding to the cell-associated form of CA 125, including antibodies shown to function to mediate lysis of CA 125-positive tumor cells. The use of radiolabeled anti-CA 125 antibodies as immunotherapeutic agents is also described in WO 2004/035537 (PCT/US2003/032945).

Ovarian cancer may respond well to platinum and taxane-based therapies, but the incidence of recurrence is high, and often ovarian tumors become resistant to these therapies (see, e.g., Vasey, Br. J. Cancer 89:S23-S28 (2003)).

An alternative approach to the treatment of cancers such as ovarian cancer has been the use of combination therapy. For example, evidence from in vitro and in vivo studies has indicated that the taxane compound paclitaxel may act as a radiosensitizer in certain cell types (see Denardo et al., Anticancer Res. 18:4011-4018 (1998); DeNardo et al., Proc. Nat. Acad. Sci. 94:4000-4004 (1997); Liebmann et al., J. Nat. Cancer Inst. 86:441-446 (1994)). Since the mechanism for the radiosensitizing effect of paclitaxel is not known, it is not possible to predict treatment regimens useful for the combined use of paclitaxel and radioimmunotherapy to treat cancers such as ovarian cancer. This is supported by an observation that the radiosensitization effect of paclitaxel shown in vitro may be highly dependent on the time of administration of paclitaxel, relative to radiotherapy (see Blumenthal et al., Anticancer Res. 23:4613-4619 (2003)).

There is a continued need for new modalities of treatment in order to decrease recurrence and improve the long-term prospects of survival for ovarian cancer patients.

Citation or identification of any reference in this or any other section of this application shall not be construed as an admission that such reference is available as prior art to the present invention.

### 4. SUMMARY OF THE INVENTION

In one aspect, the present invention provides methods for managing or treating a CA 125-related disorder, or a symptom thereof, comprising administering to a subject in need of such management or treatment a combination of: (a) a sensitizer; and (b) an antibody or an antigen-binding antibody fragment in amounts sufficient to manage or treat a CA 125-related disorder, or a symptom thereof, wherein the antibody or antigen-binding antibody fragment preferentially binds cell-associated CA 125 relative to shed CA 125.

In certain embodiments, methods of the present invention relate to management or treatment of a cell proliferative disorder or symptom thereof. In some embodiments, such methods of the present invention relate to management or treatment of a cancer or symptom thereof. In some embodiments, methods of the present invention relate to management or treatment of ovarian cancer, breast cancer, pancreatic cancer or hepatic cancer, or symptom thereof. In preferred embodiments, methods of the present invention relate to management or treatment of ovarian cancer or symptom thereof.

In certain embodiments, methods of the present invention provide for the management or treatment of a symptom of a CA 125-related disorder in a mammalian subject such as, for example, a mouse, rat, rabbit, or preferably a human.

In certain embodiments of the methods provided, the sensitizer to be administered is a taxane compound or a platinum compound. In some embodiments, the sensitizer is paclitaxel.

In some embodiments, the antibody or antigen-binding antibody fragment is conjugated to a cytotoxic agent.

In some embodiments, the cytotoxic agent is a radionuclide. Radiolabels on the antibody or antigen-binding antibody fragment to be administered can be, e.g., actinium (²²⁵Ac), astatine (²¹¹At), bismuth (²¹³Bi or ²¹²Bi), carbon (¹⁴C), cobalt (⁵⁷Co), copper (⁶⁷Cu), fluorine (¹⁸F), gallium (⁶⁸Ga or ⁶⁷Ga), holmium (¹⁶⁶Ho), indium (¹¹⁵In, ¹¹³In, ¹¹²In, or ¹¹¹In), iodine (¹³¹I, ¹²⁵I, ¹²³I, or ¹²¹I), lead (²¹²Pb), lutetium (¹⁷⁷Lu), palladium (¹⁰³Pd), phosphorous (³²P), platinum (^{195m}Pt), rhenium (¹⁸⁶Re or ¹⁸⁸Re), rhodium (¹⁰⁵Rh), ruthenium (⁹⁷Ru), samarium (¹⁵³Sm), scandium (⁴⁷Sc), technetium (^{99m}Tc), ytterbium (¹⁶⁹Yb or ¹⁷⁵Yb), or yttrium (⁹⁰Y). In certain embodiments, the radiolabel is ⁹⁰Y. The radiolabel can be conjugated to the antibody or antigen binding antibody fragment by any appropriate technique. In some embodiments, the radiolabel is linked by 1,4,7,10-tetraazacyclododecane-N,N′,N″,N′″-tetraacetic acid (DOTA) to the antibody or antigen-binding antibody fragment.

In other embodiments, the cytotoxic agent is a chemotherapeutic agent or toxin (e.g., cytostatic or cytocidal agent). Examples of chemotherapeutic agents and toxins include the following non-mutually exclusive classes: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, microbial and plant toxins, nitrosoureas, platinols, purine antimetabolites, puromycins, steroids, taxanes, topoisomerase inhibitors, and vinca alkaloids.

In some embodiments of the methods provided, the antibody to be administered is a monoclonal antibody. In other embodiments, the antigen-binding antibody fragment to be administered is an antigen-binding antibody fragment of a monoclonal antibody.

In some embodiments of the methods provided, the antibody, or antigen-binding antibody fragment to be administered binds a repeat region present within FIG. 1 (SEQ ID NO: 1).

In certain embodiments of the methods provided, the antibody or antigen-binding antibody fragment is a monoclonal antibody, or antigen-binding antibody fragment, that binds an epitope consisting of an epitope within a repeat region present within FIG. 1 (SEQ ID NO: 1). In some embodiments, the antibody or antigen-binding antibody fragment binds a non-repeat region present within SEQ ID NO: 1.

In certain embodiments, the antibody or antigen-binding antibody fragment is selected from a monoclonal antibody produced by hybridoma 4E7 (ATCC® Accession No. PTA-5109), or by hybridoma 7A11 (ATCC® Accession No. PTA-5110), or by hybridoma 7C6 (ATCC® Accession No. PTA-5111), or by hybridoma 7F10 (ATCC® Accession No. PTA-5112), or by hybridoma 7G10 (ATCC® Accession No. PTA-5245), or by hybridoma 7H1 (ATCC® Accession No. PTA-5114), or by hybridoma 8A1 (ATCC® Accession No. PTA-5115), or by hybridoma 8B5 (ATCC® Accession No. PTA-5116), or by hybridoma 8C3 (ATCC® Accession No. PTA-5246), or by hybridoma 8E3 (ATCC® Accession No. PTA-5118), or by hybridoma 8G9 (ATCC® Accession No. PTA-5119), or by hybridoma 15C9 (ATCC® Accession No. PTA-5106), or by hybridoma 16C7 (ATCC® Accession No. PTA-5107), or by hybridoma 16H9 (ATCC® Accession No. PTA-5108), or by hybridoma 117.1 (ATCC® Accession No. PTA-4567), or by hybridoma 325.1 (ATCC® Accession No. PTA-5120), or by hybridoma 368.1 (ATCC® Accession No. PTA-4568), or by hybridoma 446.1 (ATCC® Accession No. PTA-5549), or by hybridoma 501.1 (ATCC® Accession No. PTA-4569), or by hybridoma 621.1 (ATCC® Accession No. PTA-5121), or by hybridoma 633.1 (ATCC® Accession No. PTA-5122), or by hybridoma 654.1 (ATCC® Accession No. PTA-5247), or by hybridoma 725.1 (ATCC® Accession No. PTA-5124), or by hybridoma 776.1 (ATCC® Accession No. PTA-4570), or an antigen-binding antibody fragment thereof.

In certain embodiments, the antibody or antigen-binding antibody fragment is monoclonal antibody 776.1, which is produced by hybridoma 776.1 (ATCC® Accession No. PTA-4570), or an antigen-binding antibody fragment thereof.

In certain embodiments, the antibody or antigen-binding antibody fragment is selected from a monoclonal antibody or fragment thereof that competes for binding to cell-associated CA 125 with the monoclonal antibody produced by hybridoma 4E7 (ATCC® Accession No. PTA-5109), or by hybridoma 7A11 (ATCC® Accession No. PTA-5110), or by hybridoma 7C6 (ATCC® Accession No. PTA-5111), or by hybridoma 7F10 (ATCC® Accession No. PTA-5112), or by hybridoma 7G10 (ATCC® Accession No. PTA-5245), or by hybridoma 7H1 (ATCC® Accession No. PTA-5114), or by hybridoma 8A1 (ATCC® Accession No. PTA-5115), or by hybridoma 8B5 (ATCC® Accession No. PTA-5116), or by hybridoma 8C3 (ATCC® Accession No. PTA-5246), or by hybridoma 8E3 (ATCC® Accession No. PTA-5118), or by hybridoma 8G9 (ATCC® Accession No. PTA-5119), or by hybridoma 15C9 (ATCC® Accession No. PTA-5106), or by hybridoma 16C7 (ATCC® Accession No. PTA-5107), or by hybridoma 16H9 (ATCC® Accession No. PTA-5108), or by hybridoma 117.1 (ATCC® Accession No. PTA-4567), or by hybridoma 325.1 (ATCC® Accession No. PTA-5120), or by hybridoma 368.1 (ATCC® Accession No. PTA-4568), or by hybridoma 446.1 (ATCC® Accession No. PTA-5549), or by hybridoma 501.1 (ATCC® Accession No. PTA-4569), or by hybridoma 621.1 (ATCC® Accession No. PTA-5121), or by hybridoma 633.1 (ATCC® Accession No. PTA-5122), or by hybridoma 654.1 (ATCC® Accession No. PTA-5247), or by hybridoma 725.1 (ATCC® Accession No. PTA-5124), or by hybridoma 776.1 (ATCC® Accession No. PTA-4570).

In some embodiments, the methods of the present invention provide for the concurrent administration of the sensitizer with the antibody or antigen-binding antibody fragment.

In some embodiments, the sensitizer and the antibody or antibody fragment are sequentially administered. The sensitizer and the antibody or antigen-binding antibody fragment are typically administered within days, hours or minutes of each other.

In certain embodiments, the sensitizer is administered prior to administration of the antibody or antigen-binding antibody fragment.

In certain embodiments, the sensitizer is administered after the administration of the antibody or antigen-binding antibody fragment.

In another aspect, the present invention provides methods for managing or treating a symptom of a CA 125-related disorder in a subject in need thereof comprising administering to the subject a radiolabeled antibody or antigen-binding antibody fragment wherein the dose of radioactivity administered is between about 1 mCi to about 50 mCi, and wherein the radiolabeled antibody or antibody fragment preferentially binds cell-associated CA 125 polypeptide relative to shed CA 125 polypeptide. In one embodiment, the antibody or antibody fragment is labeled with [⁹⁰Y]. In another embodiment, the antibody or antibody fragment is conjugated to [⁹⁰Y-DOTA].

In another aspect, the present invention provides pharmaceutical compositions comprising: (a) a sensitizer; (b) an antibody or antigen-binding antibody fragment that preferentially binds cell-associated CA 125 polypeptide relative to shed CA 125 polypeptide; and (c) a pharmaceutically acceptable excipient, diluent, or carrier. Typically, the pharmaceutical compositions can be suitable for intravenous administration into a human. In certain embodiments the sensitizer is paclitaxel.

In yet another aspect, the present invention provides kits comprising: (a) a first pharmaceutical composition comprising a sensitizer and a pharmaceutically acceptable excipient, diluent, or carrier; and (b) a second pharmaceutical composition comprising an antibody, or an antigen-binding antibody fragment, that preferentially binds cell-associated CA 125 polypeptide relative to shed CA 125 polypeptide, and a pharmaceutically acceptable excipient, diluent, or carrier. In certain embodiments, the kits provided herein can further comprise packaging suitable for containing the first pharmaceutical composition and the second pharmaceutical composition, along with a printed label attached to or contained within the packaging describing use of the kit to treat a proliferative disease such as ovarian cancer.

4.1. Terminology

The term “about,” as used herein, unless otherwise indicated, refers to a value that is no more than 10% above or below the value being modified by the term. For example, the term “about 5 μg/kg” means a range of from 4.5 μg/kg to 5.5 μg/kg. As another example, “about 1 hour” means a range of from 54 minutes to 66 minutes.

The term “CA 125” or “CA 125 polypeptide,” as used herein, refers to the pre-shed transmembrane CA 125 polypeptide that, once shed, yields shed CA 125 polypeptide and cell-associated CA 125 polypeptide. CA 125 is also referred to in the art as 0772P or mucin-16 (or “MUC16”), which terms are avoided here in favor of the name “CA 125”, which is more widely used in the art.

The term “CA 125-related disorder,” as used herein, means a disorder that involves or is characterized by the presence of a higher level of cell-associated CA 125 relative to a corresponding normal state and/or an overabundance of shed CA 125 relative to a corresponding normal state. For example, in the case of ovarian cancer, a higher level of cell-associated or shed CA 125 is often observed relative to the level observed in a normal (e.g., non-cancerous) state. The higher level of cell-associated and/or shed CA 125 may either be causative or indicative of the disorder. In certain embodiments, a “CA 125-related disorder” according to the above is a cell proliferative disorder, typically a cancer.

As used herein, the term “cell-associated CA 125” refers to a CA 125 extracellular polypeptide species that remains in cell-associated form however transiently, e.g., prior to turn-over, after a portion of the pre-shed CA 125 polypeptide is released as shed CA 125. For example, a cell-associated CA 125 species is a CA 125 extracellular polypeptide species that remains in cell-associated form on the surface of OVCAR-3 cell line cells (American Type Culture Collection (ATCC), Manassas, Va.; product no. HTB-161) or human ascites cells after a portion of the CA 125 polypeptide is released as shed CA 125. A CA 125 cell-associated polypeptide species is present within amino acid residues 1 to 708 of SEQ ID NO:1.

As used herein, the terms “disorder” and “disease” are used interchangeably to refer to a condition in a subject.

As used herein, the term “fragment” in the phrase “antigen-binding antibody fragment” refers to a peptide or polypeptide comprising an amino acid sequence of at least about 5 contiguous amino acid residues, at least about 10 contiguous amino acid residues, at least about 15 contiguous amino acid residues, at least about 20 contiguous amino acid residues, at least about 25 contiguous amino acid residues, at least about 40 contiguous amino acid residues, at least about 50 contiguous amino acid residues, at least about 60 contiguous amino residues, at least about 70 contiguous amino acid residues, at least about 80 contiguous amino acid residues, at least about 90 contiguous amino acid residues, at least about 100 contiguous amino acid residues, at least about 110 contiguous amino acid residues, or at least about 120 contiguous amino acid residues, of the amino acid sequence of another polypeptide, e.g., an antibody that preferentially binds cell-associated CA 125.

As used herein, the terms “manage,” “managing”, “management” and the like refer to the beneficial effects that a subject suffering from a disorder derives when the methods provided herein are practiced on that subject, but which do not result in a cure of the disease. In certain embodiments, a subject is administered a combination therapy as described herein to “manage” a disorder so as to prevent or slow the progression or worsening of the disorder. For example, in some embodiments, a subject is administered a combination therapy as described herein to “manage” a disorder so as to prevent or slow tumor growth. In some embodiments, a subject is administered a combination therapy as described herein to “manage” a disorder so as to lengthen what would otherwise be the expected life span of the subject suffering from the CA 125-related disorder, but without being administered therapy for the disorder.

The term “pharmaceutically acceptable,” as used herein, refers to a composition, e.g., a carrier, excipient, or salt, approved by a regulatory agency of the Federal or a state government or listed in the U.S. Pharmacopoeia or other generally recognized pharmacopoeia for use in animals, and more particularly, in humans.

As used herein, an antibody or antigen-binding antibody fragment that “preferentially binds cell-associated CA 125,” or “preferentially binds cell-associated CA 125 polypeptide,” or “preferentially binds cell-associated CA 125 relative to shed CA 125”, or “preferentially binds CA 125 polypeptide relative to shed CA 125 polypeptide” refers to an antibody or antigen-binding antibody fragment that is positive when tested in the ELISA Competition Assay, the Flow Cytometry Competition Assay, or binds the peptide of FIG. 1, but does not detectably bind shed CA 125 polypeptide, as these assays are described in U.S. Patent Application Publication No. 2005/0064518 A1, published Mar. 24, 2005, and WO 2004/035537, published Apr. 29, 2004, which are incorporated herein by reference in their entireties for all purposes. Any antibody or antigen-binding fragment that satisfies the criteria of any one of the three previously described tests as set forth in U.S. Patent Application Publication No. 2005/0064518 A1 and WO 2004/035537 constitutes an antibody or antigen-binding fragment that “preferentially binds” cell-associated CA 125 relative to shed CA 125.

As used herein, a “protocol” includes dosing schedules and dosing regimens. The protocols herein are methods of use and include therapeutic protocols.

As used herein, a “sensitizer” is any compound that results in a fractional tumor volume (FTV) ratio of >1 as determined using the fractional product method described in Yokoyama et al., Cancer Res. 60:2190-2196 (2000), which is hereby incorporated by reference in its entirety for all purposes, when that compound is administered in any combination, that is, prior to, concurrently with, or following administration of the antibody (either alone or in association with a cytotoxic agent such as, e.g., a radionuclide (e.g., [⁹⁰Y] or non-radioactive cytotoxic agent) in the OVCAR-3 human ovarian carcinoma model described in Section 7 below. As used herein, a “sensitizer” can be any compound that fulfills the above criterion regardless of the mechanism underlying how that compound achieves its effect. By way of illustration and not limitation, a “sensitizer” can be a compound that enhances the cytotoxic effect of the naked antibody (e.g., unlabeled) alone. Alternatively, a “sensitizer” can be a radiosensitizer, for example, by enhancing the cytotoxic effect of a radiolabeled antibody (such as, e.g., [⁹⁰Y]). Alternatively, a “sensitizer” can be a compound that enhances the cytotoxic effect of the antibody conjugated to a non-radioactive cytotoxic agent.

As used herein, the term “shed CA 125 polypeptide” refers to a CA 125 extracellular polypeptide sequence that becomes separated and released from CA 125 polypeptides expressed on the surface of cells expressing CA 125, leaving a cell-associated CA 125 species remaining on the cell surface, however transiently. The term, as used herein, refers to a species of shed CA 125 found in human serum and/or OVCAR-3 (HTB-161; ATCC) cell line culture supernatant. Such shed CA 125 polypeptides can be obtained via the protocol of de los Frailes et al., Tumour Biol. 14(1):18-29 (1993), using human ascites or OVCAR-3 supernatants. Alternatively, shed CA 125 polypeptides can be obtained via commercial sources such as Fitzgerald Industries International (Concord, Mass.), Scripps Laboratories (La Jolla, Calif.), or United States Biochemical Corp (Cleveland, Ohio).

As used herein, the terms “treat”, “treatment”, “treating” and the like refer to the eradication, reduction or amelioration of a CA 125-related disorder or symptom thereof that results from the administration of a combination of sensitizer and immunotherapeutic agents as described herein. In certain embodiments, a subject is administered a combination therapy as described herein to “treat” a disorder so as to result in tumor shrinkage. In certain embodiments, a subject is administered a combination therapy as described herein to “treat” a disorder so as to prevent or halt spread of a cancer.

### 5. BRIEF DESCRIPTION OF THE DRAWINGS

FIG. 1: Depicts the polypeptide, designated as CA 125 3-repeat (SEQ ID NO: 1), that includes the extracellular portion of the CA 125 amino acid sequence representing the three carboxyl-most repeat domains up to, but not including, the CA 125 transmembrane sequence. Italicized residues from amino acid 14 to amino acid 452 represent repeat regions. Each of the three repeats within the 14-452 repeat region are delineated by vertical lines and arrows as shown. Underlined residues represent the transmembrane-proximal non-repeat region. The sequence that follows the underlined residues is not part of CA 125 and includes a carboxy-Myc-His tag.

FIG. 2: Effect of combined therapy using paclitaxel and low-dose [⁹⁰Y-DOTA]776.1 on the growth of OVCAR-3 tumors and comparison with paclitaxel monotherapy and with [⁹⁰Y-DOTA]776.1 monotherapy.

FIG. 3: Effect of dosing strategy on the growth of OVCAR-3 tumors.

FIG. 4: Effect of combined therapy using paclitaxel and high-dose [⁹⁰Y-DOTA]776.1 on the growth of OVCAR-3 tumors and comparison with monotherapies.

FIG. 5: Effect of combination therapy using low-dose radioimmunotherapy and paclitaxel on weight gain in mice receiving treatment.

### 6. DETAILED DESCRIPTION OF THE INVENTION

The present invention is based, in part, on the observation of a synergistic effect arising from combined administration of paclitaxel with [⁹⁰Y] 776.1, which is a radiolabeled monoclonal antibody that preferentially binds cell-associated CA 125 relative to shed CA 125, on the reduction or regression of tumor growth in an aggressive xenograft model of human ovarian cancer, as demonstrated in the working Examples below. The methods of the present invention are particularly useful in that the broad toxic side effects of extended paclitaxel monotherapy or of high doses of radioactivity in the radioimmunotherapeutic agent can be ameliorated or avoided by performing the methods provided herein.

In one aspect the present invention provides methods of managing or treating a CA 125-related disorder, or symptom thereof, in a subject in need thereof comprising administering to the subject a combination of a sensitizer and an antibody or antigen-binding antibody fragment that preferentially binds cell-associated CA 125 relative to shed CA 125.

6.1. Indications

In certain embodiments, the present invention provides methods relating to the management or treatment of a CA 125-related disorder or symptom thereof in a subject in need of such management or treatment.

In certain embodiments, methods of the present invention relate to management or treatment of an ovarian cancer, breast cancer, pancreatic cancer or hepatic cancer, or symptom thereof.

In preferred embodiments, the methods of the present invention relate to the management or treatment of an ovarian cancer or symptom thereof.

In certain embodiments, the methods of the present invention can be used to manage or treat a CA 125-related disorder in a cancer or tumor cell expressing a mutated p53 gene.

As used herein, the terms “subject” and “patient” are used interchangeably, and can refer to an animal, preferably a mammal (e.g., a mouse, rat, guinea pig, rabbit, cow, pig, horse, donkey, goat, sheep, camel, cat, dog), and more preferably a primate (e.g., a monkey, such as a cynomolgous monkey, gorilla, chimpanzee), and most preferably a human.

6.2. Antibodies and Antigen-Binding Antibody Fragments

The antibody or antigen-binding antibody fragment to be administered in the methods provided herein are those that preferentially bind cell-associated CA 125 such as, for example, those described in U.S. Patent Application Publication No. 2005/0064518 A1, published Mar. 24, 2005, and WO 2004/035537, published Apr. 29, 2004, which are incorporated herein by reference in their entireties. Due to the fact that cell-associated CA 125, prior to CA 125 shedding, is present as part of pre-shed CA 125, the skilled artisan will recognize that antibodies and antigen-binding antibody fragments that preferentially bind cell-associated CA 125 can also bind pre-shed CA 125. Thus, while not wishing to be bound by any particular mechanism or theory thereof, it is noted that the methods described herein can be effectuated by binding of the administered antibody or antigen-binding antibody fragment to pre-shed CA 125 or to cell-associated CA 125, or to both.

In certain embodiments, the antibody or antigen-binding antibody fragment is a monoclonal antibody or antigen-binding antibody fragment thereof, respectively.

In certain embodiments, the antibody or antigen-binding antibody fragment is selected from a monoclonal antibody produced by hybridoma 4E7 (ATCC® Accession No. PTA-5109), or by hybridoma 7A11 (ATCC® Accession No. PTA-5110), or by hybridoma 7C6 (ATCC® Accession No. PTA-5111), or by hybridoma 7F10 (ATCC® Accession No. PTA-5112), or by hybridoma 7G10 (ATCC® Accession No. PTA-5245), or by hybridoma 7H1 (ATCC® Accession No. PTA-5114), or by hybridoma 8A1 (ATCC® Accession No. PTA-5115), or by hybridoma 8B5 (ATCC® Accession No. PTA-5116), or by hybridoma 8C3 (ATCC® Accession No. PTA-5246), or by hybridoma 8E3 (ATCC® Accession No. PTA-5118), or by hybridoma 8G9 (ATCC® Accession No. PTA-5119), or by hybridoma 15C9 (ATCC® Accession No. PTA-5106), or by hybridoma 16C7 (ATCC® Accession No. PTA-5107), or by hybridoma 16H9 (ATCC® Accession No. PTA-5108), or by hybridoma 117.1 (ATCC® Accession No. PTA-4567), or by hybridoma 325.1 (ATCC® Accession No. PTA-5120), or by hybridoma 368.1 (ATCC® Accession No. PTA-4568), or by hybridoma 446.1 (ATCC® Accession No. PTA-5549), or by hybridoma 501.1 (ATCC® Accession No. PTA-4569), or by hybridoma 621.1 (ATCC® Accession No. PTA-5121), or by hybridoma 633.1 (ATCC® Accession No. PTA-5122), or by hybridoma 654.1 (ATCC® Accession No. PTA-5247), or by hybridoma 725.1 (ATCC® Accession No. PTA-5124), or by hybridoma 776.1 (ATCC® Accession No. PTA-4570), or an antigen-binding antibody fragment thereof.

In certain embodiments, the antibody or antigen-binding antibody fragment is monoclonal antibody 776.1 produced by hybridoma 776.1 (ATCC® Accession No. PTA-4570) or an antigen-binding antibody fragment thereof.

In certain embodiments, the antibody or antigen-binding antibody fragment is selected from a monoclonal antibody or antigen-binding antibody fragment thereof that competes for binding to cell-associated CA 125 with the monoclonal antibody produced by hybridoma 4E7 (ATCC® Accession No. PTA-5109), or by hybridoma 7A11 (ATCC® Accession No. PTA-5110), or by hybridoma 7C6 (ATCC® Accession No. PTA-5111), or by hybridoma 7F10 (ATCC® Accession No. PTA-5112), or by hybridoma 7G10 (ATCC® Accession No. PTA-5245), or by hybridoma 7H1 (ATCC® Accession No. PTA-5114), or by hybridoma 8A1 (ATCC® Accession No. PTA-5115), or by hybridoma 8B5 (ATCC® Accession No. PTA-5116), or by hybridoma 8C3 (ATCC® Accession No. PTA-5246), or by hybridoma 8E3 (ATCC® Accession No. PTA-5118), or by hybridoma 8G9 (ATCC® Accession No. PTA-5119), or by hybridoma 15C9 (ATCC® Accession No. PTA-5106), or by hybridoma 16C7 (ATCC® Accession No. PTA-5107), or by hybridoma 16H9 (ATCC® Accession No. PTA-5108), or by hybridoma 117.1 (ATCC® Accession No. PTA-4567), or by hybridoma 325.1 (ATCC® Accession No. PTA-5120), or by hybridoma 368.1 (ATCC® Accession No. PTA-4568), or by hybridoma 446.1 (ATCC® Accession No. PTA-5549), or by hybridoma 501.1 (ATCC® Accession No. PTA-4569), or by hybridoma 621.1 (ATCC® Accession No. PTA-5121), or by hybridoma 633.1 (ATCC® Accession No. PTA-5122), or by hybridoma 654.1 (ATCC® Accession No. PTA-5247), or by hybridoma 725.1 (ATCC® Accession No. PTA-5124), or by hybridoma 776.1 (ATCC® Accession No. PTA-4570).

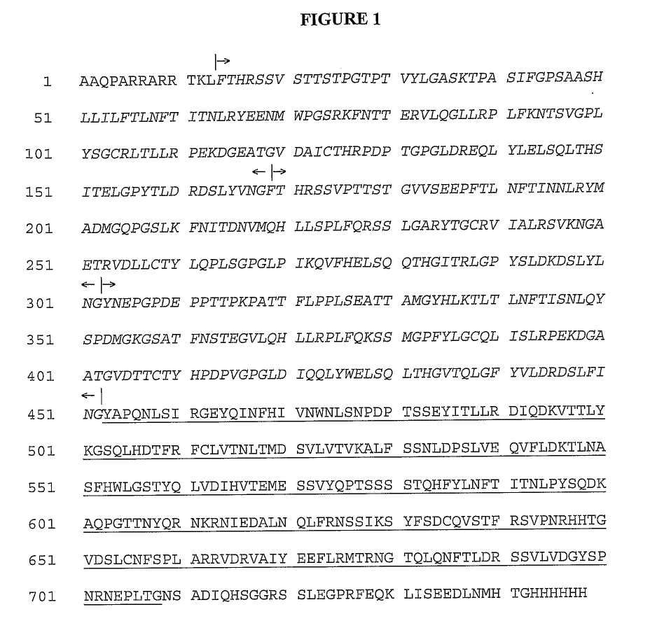

In certain embodiments, the antibody or antigen-binding antibody fragment to be administered binds a repeat region present within FIG. 1 (SEQ ID NO:1).

In certain embodiments, the antibody or antigen-binding antibody fragment is a monoclonal antibody or antigen-binding monoclonal antibody fragment that binds an epitope within a repeat region present within FIG. 1 (SEQ ID NO:1).

In some embodiments, the antibody or antigen-binding antibody fragment binds a non-repeat region present within SEQ ID NO:1.

Antibodies or antigen-binding antibody fragments to be administered can be antibodies or antigen-binding antibody fragments, respectively, that inhibit CA 125-positive tumor growth either by themselves (i.e., as naked antibodies or naked fragments), or conjugated to a cytotoxic agent. For example, such antibodies or antigen-binding antibody fragments are those that inhibit CA 125-positive tumor growth in such animal models as those described in Treskes et al., Eur. J. Cancer. 30A(2):183-187 (1994); Ahmad et al., Oncol. Res. 11(6):273-280 (1999); and Kievit et al., Int. J. Radiat. Oncol. Biol. Phys. 38(2):419-428 (1997); or in the OVCAR-3 xenograft tumor animal model described below.

In certain embodiments, the antibody or antigen-binding antibody fragment to be administered is associated with (i.e., conjugated to) a cytotoxic agent. The cytotoxic agent can be any agent known in the art that can be conjugated, directly or indirectly, covalently or non-covalently, to an antibody or antigen-binding antibody fragment, and that can be used in managing or treating a CA 125-related disorder (e.g., a cancer) or symptom thereof in a subject.

As used herein, a “labeled” antibody or “labeled” antigen-binding antibody fragment means an antibody or antigen-binding antibody fragment that is associated with a cytotoxic agent either directly or through a linker moiety. For example, a “radiolabeled” antibody is an antibody associated with a radionuclide.

In certain embodiments of the methods provided, the antibody or antigen-binding antibody fragment is a radiolabeled antibody or radiolabeled antigen-binding antibody fragment. In general, the radiolabel can be any radionuclide known in the art to be potentially useful for radiation cancer therapy. Radiolabels on the antibody or antigen-binding antibody fragment to be administered can be, for example, actinium (²²⁵Ac), astatine (²¹¹At), bismuth (²¹³Bi or ²¹²Bi), carbon (¹⁴C), cobalt (⁵⁷Co), copper (⁶⁷Cu), fluorine (¹⁸F), gallium (⁶⁸Ga or ⁶⁷Ga), holmium (¹⁶⁶Ho), indium (¹¹⁵In, ¹¹³In, ¹¹²In or ¹¹¹In), iodine (¹³¹I, ¹²⁵I, ¹²³I or ¹²¹I), lead (²¹²Pb), lutetium (¹⁷⁷Lu), palladium (¹⁰³Pd), phosphorous (³²P), platinum (^{195m}Pt), rhenium (¹⁸⁶Re or ¹⁸⁸Re), rhodium (¹⁰⁵Rh), ruthenium (⁹⁷Ru), samarium (¹⁵³Sm), scandium (⁴⁷Sc), technietium (^{99m}Tc), ytterbium (¹⁶⁹Yb or ¹⁷⁵Yb), or yttrium (⁹⁰Y), and so forth, without limitation. In certain embodiments, the radiolabel is ⁹⁰Y.

The radiolabel can be associated with the antibody or antigen-binding antibody fragment by any means known in the art. For example, direct radio-iodinization (with ¹³¹I, ¹²⁵I, or ¹²³I) of antibodies is generally well established. Bifunctional chelating agents for attaching metallic radionuclides to antibodies or antigen-binding antibody fragments can be any of those known in the art. Such chelating agents can be, e.g., a diethylenetriamine pentaacetic acid (DTPA) compound such as cyclohexyl-DTPA or MX-DTPA, or 1,4,7,10-tetraazacyclododecane-N,N′,N″,N′″-tetraacetic acid (DOTA).

In certain embodiments, the radiolabeled antibody or antigen-binding antibody fragment is a DOTA-conjugated antibody or DOTA-conjugated antigen-binding antibody fragment. For example, a ⁹⁰Y-conjugated antibody can be a [⁹⁰Y-DOTA] antibody.

In certain embodiments wherein a radiolabeled antibody or radiolabeled antigen-binding antibody fragment is administered, the specific activity and radioactive dose to be administered can be determined in accordance with criteria known in the art, and depending upon such factors as the particular radiolabel, the weight, sex, age and general condition of the subject, the protocol of administration, and so forth. The dose can also be adjusted in view of the response to therapy.

In a non-limiting embodiment of the methods provided wherein a ⁹⁰Y-radiolabeled mAb or fragment is administered, the radioactive dose administered can be between about 1 mCi and about 50 mCi, or between about 1 mCi and about 40 mCi, or between about 5 mCi and about 30 mCi or between about 10 mCi and about 20 mCi.

In certain embodiments of the methods provided, the antibody or antigen-binding antibody fragment is conjugated to a cytotoxic agent other than a radionuclide. Such a cytotoxic agent can be, for example, a chemotherapeutic agent or toxin (e.g., cytostatic or cytocidal agent). Examples of chemotherapeutic agents and toxins include the following non-mutually exclusive classes: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, microbial toxins, plant toxins, nitrosoureas, platinols, purine antimetabolites, puromycins, steroids, taxanes, topoisomerase inhibitors, and vinca alkaloids. Examples of individual chemotherapeutics or toxins that can be conjugated to the antibody or antigen-binding antibody fragment include but are not limited to an abrin, androgen, anthramycin (AMC), asparaginase, auristatin E, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, fluosol, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, plicamycin, procarbizine, pseudomonas exotoxin, ricin A, streptozotocin, tenoposide, 6-thioguanine, thioTEPA, topotecan, triapazamine, vinblastine, vincristine, vinorelbine, VP-16 and VM-26.

Typically, the antibody or antigen-binding antibody fragment to be administered in the methods provided is administered as a pharmaceutical composition comprising the antibody or antigen-binding antibody fragment and a pharmaceutically acceptable carrier. The term “carrier” refers to a diluent, excipient, stabilizing agent, preservative, binder, or vehicle, or a combination thereof, adapted for administration of the antibody or antigen-binding antibody fragment.

Pharmaceutical carriers can be sterile liquids, such as water and oils, including those of petroleum, animal, vegetable or synthetic origin, such as peanut oil, soybean oil, mineral oil, sesame oil and the like. Water, saline solutions and aqueous dextrose, sucrose and glycerol solutions can also be employed as liquid carriers, particularly for injectable solutions.

In a preferred embodiment, the pharmaceutical composition is sterile and in suitable form for administration to a subject, preferably to an animal subject, more preferably to a mammalian subject, and most preferably to a human subject.

In the methods provided, the antibody or antigen-binding antibody fragment can be administered to a subject at dosage concentration (mass polypeptide per subject body mass) of from about 5 μg/kg to about 10 mg/kg, more preferably from about 20 μg/kg to about 5 mg/kg, and most preferably from about 100 μg/kg to about 5 mg/kg.

The precise dose to be employed in the formulation will typically depend on the route of administration, and the seriousness of the condition, and may be decided according to the judgment of the practitioner and each patient's circumstances in view of published clinical studies. Effective doses can be extrapolated from dose-response curves derived from in vitro or animal model test systems.

6.3. Sensitizers

The sensitizer to be administered to the subject in the methods provided herein can be any sensitizer according to the definition of “sensitizer” provided above. For example, a compound useful as a sensitizer can be selected from: (a) an antitumor antibiotic such as, e.g., acivicin, actinomycin D, bleomycin, daunorubicin, doxorubicin, plicamycin, and so forth; (b) a platinum-containing compound such as, e.g., cisplatin, carboplatin, or oxaliplatin; (c) a substituted urea or nitrosourea such as, e.g., hydroxyurea, carmustine, lomusitne, semustine, or streptozocin; (d) an antimetabolite, such as, e.g., a folic acid analog (e.g., methotrexate), a pyrimidine analog (e.g., cytarabine), or a purine analog (e.g., mercaptopurine, thioguanine, pentostatin); (e) a vinca alkaloid such as, e.g., vinblastin or vincristine; (f) a nitrogen mustard such as, e.g., mechloroethamine, cyclophosphamide, chlorambucil, or melphalan; and (g) a taxane such as, e.g., paclitaxel or docetaxol.

In certain embodiments, the sensitizer can be: (a) an agent that stabilizes microtubule formation; (b) an agent that blocks cells at the G2/M stage; (c) an agent that upregulates bcl-2 phosphorylation; or (d) a radiosensitizer.

Typically, the sensitizer is administered as part of a pharmaceutical composition further comprising a pharmaceutically acceptable carrier.

Doses of the sensitizer to be administered will depend upon the characteristics of the particular sensitizer used. Those of skill in the art will be guided by typical doses and protocols already known in the art for the administration of the sensitizer in view, inter alia, of the particular patient and disorder to be treated. However, it will be further recognized in view of the synergistic effect provided by the combined administration of sensitizer and antibody or antigen-binding antibody fragment, as exemplified in the Examples below, that the dose and/or frequency of administration of the sensitizer will generally be in lower amounts or less frequent than in monotherapy.

In preferred embodiments, the sensitizer is paclitaxel. Paclitaxel is available under the tradename TAXOL® (Bristol-Meyers-Squibb, Princeton N.J.)), as an injectable solution. It is supplied as a nonaqueous solution intended for dilution with a suitable parenteral fluid prior to intravenous infusion. TAXOL® is available in 30 mg (5 mL), 100 mg (16.7 mL), and 300 mg (50 mL) multidose vials. Each mL of sterile nonpyrogenic solution contains 6 mg paclitaxel, 527 mg of purified Cremophor EL (polyoxyethylated castor oil) and 49.7% (v/v) dehydrated alcohol, USP. Paclitaxel can be diluted with 0.9% sodium chloride injection, USP, 5% dextrose injection, USP, 5% dextrose and 0.9% sodium chloride injection, USP, or 5% dextrose in Ringers injection to a final concentration of 0.3-1.2 mg/mL. Dosing regimens for paclitaxel include those described in the Physicians Desk Reference (2000), which is incorporated herein by reference in its entirety for all purposes.

6.4. Combination Administration Methods

In general, the methods described herein can be utilized by administration to a subject of a sensitizer in combination with an antibody or antigen-binding antibody fragment as described above.

The efficacy and toxicity of the combination treatment with sensitizer and antibody or antigen-binding antibody fragment administered according to the particular protocols practiced as part of the present invention can be determined by standard pharmaceutical procedures in cell cultures or experimental animals, e.g., for determining the ED₅₀ (the dose therapeutically effective in 50% of the population) and the LD₅₀ (the dose lethal to 50% of the population). Compositions that exhibit large therapeutic indices are preferred.

Data obtained from cell culture assays and animal studies can be used in formulating a range of doses of sensitizer and antibody (or antigen-binding antibody fragment) for use in human subjects. The doses of such components are preferably within a range that results in circulating concentrations that include the ED₅₀ with little or no toxicity. Dosages may vary within this range depending upon the dosage form employed and the route of administration utilized. For any agent used in the methods of the present invention, the therapeutically effective dose can be estimated initially from cell culture assays. A dose may be formulated in animal models to achieve a circulating plasma concentration range that includes the IC₅₀ (i.e., the concentration of the compound that achieves a half-maximal inhibition of one or more symptoms) as determined in cell culture assays, e.g., proliferation assays. Such information can be used to more accurately determine useful doses in humans. Levels in plasma may be measured, e.g., by high performance liquid chromatography.

In a specific embodiment, it may be desirable to administer the sensitizer and antibody or antigen-binding antibody fragment either systemically or locally to the area in need of treatment. Under some circumstances, it may be necessary to use a composition exhibiting toxic side effects. In such circumstances, it may be preferable to deliver such a composition directly to the site of the affected tissue, e.g., ovarian cancer tissue, thereby helping to reduce potential systemic side effects.

This can be achieved, e.g., by local infusion, injection, or by means of an implant, said implant being of a porous, non-porous, or gelatinous material, including membranes, such as sialastic membranes, or fibers. Preferably, when administering a pharmaceutical composition, care will be taken to use materials to which the sensitizer and antibodies or antigen-binding antibody fragments do not adsorb.

The sensitizer and the antibody or antigen-binding antibody fragment can be administered by the same or by different methods. Routes of administration can include, but are not limited to, parenteral (e.g., intradermal, intramuscular, intraperitoneal, intravenous and subcutaneous administration), epidural, or mucosal (e.g., intranasal and oral) routes of administration. See, e.g., U.S. Pat. Nos. 5,679,377; 5,702,727; 5,783,193; 5,817,624; 6,074,689; 6,156,731; 6,174,529; 6,187,803; 6,331,175; and 6,387,406. In a specific embodiment, either the sensitizer or the antibody (or antigen-binding antibody fragment) is administered intramuscularly, intraperitoneally, intravenously, or subcutaneously. In another specific embodiment, both the sensitizer and the antibody (or antigen-binding antibody fragment) are administered intramuscularly, intraperitoneally, intravenously, or subcutaneously.

In certain embodiments, both the sensitizer and the antibody (or antigen-binding antibody fragment) are formulated in pharmaceutically acceptable compositions in accordance with routine procedures so that each composition is adapted for intravenous administration to a human subject. Typically, pharmaceutical compositions for intravenous administration are solutions in sterile isotonic aqueous buffer. Where necessary, a local anesthetic can be administered at the site of the injection to ease pain.

The use of the term “combination therapy” or “combination cancer therapy” does not limit the order in which agents or treatments are administered to a subject having a CA 125-related disorder. For example, the agents of the combination therapy can be administered concurrently, sequentially in any order or cyclically to a subject. In a preferred embodiment, the two components of the combination therapy are administered concurrently to a subject.

The two components of the combination therapy can be administered to a subject in the same pharmaceutical composition. Alternatively, the two components of the combination therapies can be administered to a subject in two separate pharmaceutical compositions, and these two compositions may be administered by the same or by different routes of administration.

In certain embodiments, the sensitizer and antibody (or antigen-binding antibody fragment) are sequentially administered. The sensitizer and antibody (or antigen-binding antibody fragment) may be administered within days, hours or minutes of each other.

In certain embodiments, the sensitizer is administered about 1 hour, about 5 hours, about 8 hours, about 10 hours, about 15 hours, about 20 hours, about 24 hours, about 30 hours, about 36 hours, about 42 hours, about 48 hours, or about 54 hours before or after administration of the antibody or antigen-binding antibody fragment. In other embodiments, the sensitizer is administered up to about 7 days before or after administration of the antibody or antigen-binding antibody fragment.

A single dose of either or both components can be a continuous administration by infusion or injection over a period of time, e.g., from a few seconds to over 24 hours. Multiple doses of either or both components can be administered. For example, single doses of each component can be administered over a period of about 1 day up to about 6 days, about weekly, about every two weeks, about every three weeks, about every four weeks, about every five weeks, or about every six weeks.

In certain preferred embodiments, the sensitizer is paclitaxel, which is administered prior to the administration of antibody or antigen-binding antibody fragment. As shown in the Examples below, reduced toxicity is observed when using a dosing strategy in which paclitaxel is administered prior to administration of radiomimunotherapy.

6.5. High Dose Radioimmunotherapy Administration Methods

In another aspect, the present invention provides methods for managing or treating a CA 125-related disorder in a subject in need thereof comprising administering to the subject a radiolabeled (e.g., a [⁹⁰Y]-labeled) antibody or antigen-binding antibody fragment such that the cell proliferative disorder is managed or treated, wherein the antibody or antigen-binding antibody fragment preferentially binds cell-associated CA 125 relative to shed CA 125 polypeptide, and wherein the radioactive dose administered is between about 1 mCi and 50 mCi.

In certain embodiments, the radiolabeled antibody is a monoclonal antibody (mAb). The amount (μg) of mAb administered per subject body mass can be, e.g., in an amount described in Section 6.2 above. In certain embodiments, the radiolabeled antibody is a [⁹⁰Y]mAb. In certain embodiments, the [⁹⁰Y]mAb is a [⁹⁰Y]-776.1 antibody. In certain embodiments, the radiolabeled antibody is a [⁹⁰Y-DOTA]mAb. In certain embodiments, the [⁹⁰Y-DOTA]mAb is a [⁹⁰Y-DOTA]-776.1 antibody.

6.6. Kits

In another aspect, the present invention provides kits comprising: (a) a first pharmaceutical composition comprising a sensitizer and a pharmaceutically acceptable excipient, diluent, or carrier; and (b) a second pharmaceutical composition comprising an antibody, or an antigen-binding antibody fragment, that preferentially binds cell-associated CA 125 polypeptide relative to shed CA 125 polypeptide, and a second pharmaceutically acceptable excipient, diluent, or carrier.

In certain embodiments, the kit can further comprise packaging materials suitable for containing the pharmaceutical compositions therein, along with a printed label attached to or contained within the packaging, describing use of the pharmaceutical compositions to treat a cancer such as ovarian cancer, breast cancer, pancreatic cancer or hepatic cancer.

In certain embodiments of the kits provided, the first pharmaceutical composition and the second pharmaceutical composition can comprise a single mixture. In certain embodiments of the kits provided, the first pharmaceutical composition and the second pharmaceutical composition can comprise separate mixtures. When appropriate, the compositions for injection can be presented in unit dosage forms such as, e.g., in ampoules or in multidose containers, and can optionally further comprise an added preservative.

### 7. EXAMPLES

The results provided herein demonstrate that tumor growth in an art-recognized murine xenograft model of human ovarian cancer can be slowed or reversed by combination therapy using paclitaxel and low-dose [⁹⁰Y-DOTA]776.1 antibody.

Model of Human Cancer: A subcutaneous OVCAR-3 xenograft model was used. The OVCAR-3 cell line can easily be grown as subcutaneous tumors in immunocompromised mice; however, tumor take and growth kinetics were enhanced by passaging as intraperitoneal tumors prior to subcutaneous implantation, as described below.

Animals: Female NCr nu/nu (“nude”) mice (Taconic Farms, Germantown, N.Y.) 6-7 weeks old were used for all studies. All animals were housed in sterile micro-isolators, and were given autoclaved food and water ad libitum.

Tumor Cell Implantation: The OVCAR-3 human ovarian carcinoma cell line was used as an art-recognized model of human ovarian cancer (see Hamilton et al., Cancer Res. 43:5379-5389 (1983); Burbridge et al., Int. J. Oncol. 15:1155-1162 (1999)). The OVCAR-3 cell line, derived from a human ovarian adenocarcinoma, was purchased from the ATCC (NIH: OVCAR-3, Catalog #HTB-161). OVCAR-3 cells express the tumor-associated CA 125 on the cell surface. OVCAR-3 cells were maintained in RPMI-1640 supplemented with 10% FBS at 37° C. in 5% CO₂. OVCAR-3 cells were serially propagated in vivo 3 times within the peritoneal cavities of NCr nu/nu mice, followed by subcutaneous implantation in the flank of the mice. For subcutaneous implantation, cells were resuspended to a final concentration of 15×10⁶ cells/ml in a mixture of Matrigel (Matrigel, BD Biosciences, Chicago, Ill.: Lot #005002, 14.6 mg/ml) and 0.9% saline. Mice were injected with 0.2 ml volume of the cell suspension for a final dose of 3×10⁶ cells. Beginning approximately 10 days post-implantation, palpable tumors were measured with electronic calipers (Fowler Instruments, Newton, Mass.) across two perpendicular dimensions. Mice were randomly sorted into groups of eight based on tumor volume. Tumor volumes at the start of treatment ranged from 150 mm³ to 350 mm³. For all groups within a study, there were no significant differences between mean starting tumor volumes. Tumor measurements and observations were recorded twice a week. Tumor volume was calculated using the standard formula (Length×Width²)×0.5.

7.1. Antibody Generation: Preparation of [⁹⁰Y-DOTA]776.1

Methods of producing antibodies and antigen-binding antibody fragments, including chimeric, radiolabeled and/or humanized forms thereof, that preferentially bind cell-associated CA 125 relative to shed CA 125 are described in U.S. Patent Application Publication No. 2005/0064518 A1, published Mar. 24, 2005, and WO 2004/035537, published Apr. 29, 2004, which are both incorporated herein by reference in their entireties for all purposes. These references also provide several methods, such as an ELISA competition assay, a flow cytometry competition assay, Western blot techniques, and an affinity assay for demonstrating the specificity and high degree of affinity for cell-associated CA 125 that characterizes the 776.1 monoclonal antibody, among other antibodies disclosed therein that preferentially bind cell associated CA 125. U.S. Patent Application Publication No. 2005/0064518 A1 and WO 2004/035537 also describe functional assays demonstrating lysis of CA 125-positive tumor cells mediated by the 776.1 antibody; the amino acid sequence of the 776.1 antibody; and the utility of ¹³¹I- or ⁹⁰Y-radiolabeled 776.1 antibodies in single-dose radioimmunotherapy in a murine xenograft model of human ovarian cancer.

The following hybridomas have been deposited with the American Type Culture Collection (ATCC), P.O. Box 1549, Manassas, Va. 20108 USA, under the provisions of the Budapest Treaty on the International Recognition of the Deposit of Microorganisms for the Purpose of Patent Procedures: hybridoma 4E7 (ATCC® Accession No. PTA-5109); hybridoma 7A11 (ATCC® Accession No. PTA-5110); hybridoma 7C6 (ATCC® Accession No. PTA-5111); hybridoma 7F10 (ATCC® Accession No. PTA-5112); hybridoma 7G10 (ATCC® Accession No. PTA-5245); hybridoma 7H1 (ATCC® Accession No. PTA-5114); hybridoma 8A1 (ATCC® Accession No. PTA-5115); hybridoma 8B5 (ATCC® Accession No. PTA-5116); hybridoma 8C3 (ATCC® Accession No. PTA-5246); hybridoma 8E3 (ATCC® Accession No. PTA-5118); hybridoma 8G9 (ATCC® Accession No. PTA-5119); hybridoma 15C9 (ATCC® Accession No. PTA-5106); hybridoma 16C7 (ATCC® Accession No. PTA-5107); hybridoma 16H9 (ATCC® Accession No. PTA-5108); hybridoma 117.1 (ATCC® Accession No. PTA-4567); hybridoma 325.1 (ATCC® Accession No. PTA-5120); hybridoma 368.1 (ATCC® Accession No. PTA-4568); hybridoma 446.1 (ATCC® Accession No. PTA-5549); hybridoma 501.1 (ATCC® Accession No. PTA-4569); hybridoma 621.1 (ATCC® Accession No. PTA-5121); hybridoma 633.1 (ATCC® Accession No. PTA-5122); hybridoma 654.1 (ATCC® Accession No. PTA-5247); hybridoma 725.1 (ATCC® Accession No. PTA-5124); and hybridoma 776.1 (ATCC® Accession No. PTA-4570).

Isolation of MAb 776.1: Hybridoma 776.1, expressing murine IgG1 776.1, was isolated from a splenic fusion of mice immunized initially with OVCAR-3 cells, followed by boosts with a recombinant fragment of CA 125 using standard immunization and hybridoma techniques. The 776.1 monoclonal antibody was isolated from hybridoma supernatants by purification on protein A columns (Amersham-Pharmacia, Piscataway, N.J.).

Coupling of MAb 776.1 to ⁹⁰Yttrium: MAb 776.1 was conjugated with DOTA and then labeled with yttrium-90 using a modification of the methods of Lewis et al., Bioconjugate Chem. 5:565-576 (1994) and Lewis et al., Bioconjugate Chem. 12:320-324 (2001), as described below.

Ten milligrams of either purified murine IgG1 776.1 or MOPC-21 (control IgG1; Sigma, St. Louis, Mo.) at 10 mg/ml were dialyzed against 2 L of 0.1 M NaHCO₃/0.1 M K₂HPO₄, pH 8.5, over Chelex-100 (Bio-Rad Laboratories, Hercules, Calif.) at 4° C. for 48 hr. with one buffer change. Protein recovery was determined using a Bradford assay.

MAb 776.1 was conjugated with DOTA through lysine residues using the bifunctional chelator DOTA-NHS (1,4,7,10-tetraazacyclododecane-1,4,7,10-tetraacetic acid mono(N-hydroxysuccinimidyl ester)). A 5-molar excess of DOTA-NHS (Macrocyclics, Dallas, Tex.) dissolved in metal-free H₂O was added to the antibody and incubated on a rotator O/N at 4° C. The DOTA-conjugated antibody was then dialyzed against 2 L of 0.1 M NaHCO₃/0.1 M K₂HPO₄, pH 8.5, over Chelex-100 at 4° C. O/N to remove unconjugated DOTA-NHS, then subsequently against 2 L of 0.25 M NH₄OAc, pH 6.0 over Chelex-100 for 72 hr. with two buffer changes. The total recovery of [DOTA]776.1 was determined by the Bradford assay. The number of DOTA residues per antibody was determined by the Arsenazo III/Pb assay (see Dadachova et al., Nucl. Med. Biol. 26:977-982 (1999)).

For labeling of the DOTA-776.1 with ⁹⁰Y, 1 to 1.5 mg DOTA-776.1 were diluted to 5 mg/ml in 0.25 M NH₄OAc, pH 6.0, and freshly-prepared ascorbic acid (from 1 M stock, pH 5.0) was added to a final concentration of 10 mg/ml. The mixture was prewarmed for 5 min at 40° C., and then 5-10 mCi ⁹⁰YCl₃ (200 mCi/ml in 0.05 N HCl (MDS-Nordion, Ottawa, ON)) was added, mixed, and incubated at 40° C. for 1 hr. The reaction was terminated by the addition of DTPA to a final concentration of 1 mM to chelate any remaining free ⁹⁰Y and incubated at room temperature for 10 min. Percent incorporation was determined by ITLC. Unincorporated ⁹⁰Y and ascorbic acid were removed by separation on NAP-10 G-25 columns pre-equilibrated with 2×5 ml 0.9% saline containing 0.1% mouse serum albumin (Sigma, St. Louis, Mo.). The saline/MSA buffer was used as running buffer. Percent unincorporated ⁹⁰Y of the final preparation was determined by ITLC. The final preparation was diluted to appropriate dosing concentrations with 0.9% saline and sterile-filtered into injection vials.

Prior to use, all glassware to be used for conjugation and labeling of antibodies was soaked in 10% nitric acid at least overnight and rinsed thoroughly in Milli-Q-grade (or higher grade) water. All plasticware, including tubes, were either purchased as certified metal-free (i.e., no release agents used), or soaked in 10% nitric acid at least overnight and thoroughly rinsed. Microfuge tubes used for conjugation, storing of antibody, or in labeling were rinsed with water treated with Chelex-100. All solutions used including dialysis solutions were treated by passing down a Chelex-100 column prior to use and stored in acid-soaked glassware.

Following the protocols described above, the average DOTA:mAb ratio was 5.2:1. [DOTA]776.1 was labeled with ⁹⁰yttrium to a specific activity of 8.21 mCi/mg, and the immunoreactivity of [⁹⁰Y-DOTA]776.1 at the time of treatment was >95%.

Immunoreactivity of [⁹⁰Y-DOTA]776.1: The immunoreactivity of radiolabeled 776.1 was determined by ELISA assay. Immunlon 4 (Dynatech, Chantilly, Va.) 96-well plates were coated with 100 μl per well of a recombinant, HA-tagged, soluble form of CA 125 representing the three most C-terminal tandem repeats, as well as a non-repetitive domain between these repeats and the transmembrane domain (as shown in FIG. 1) at 1 μg/ml in DPBS overnight at 4° C. The next day, the plates were blocked with 200 μl per well of blocking buffer (1×PBS with 1% BSA) for 1 hour at room temperature. Unlabeled and radiolabeled 776.1 were diluted to 3 μg/ml in blocking buffer and added to the first row of the blocked plate in triplicate at 150 μl per well; 100 μl of blocking buffer was added to the remaining wells. Antibodies were then serially diluted. The plate was incubated for 1 hour at room temperature, followed by three washes with DPBS containing 0.05% Tween-20 (PBST; 200 μl per well). For signal detection, 100 μl of HRP-conjugated goat anti-mouse IgG (Amersham Biosciences, Piscataway, N.J., diluted 1:2000) was added to each well and incubated for 1 hour at room temperature. The plates were washed three times with PBST and the HRP conjugate was detected by adding a mixture of TMB substrate and H₂O₂ (1:1 ratio, KPL, Gaithersburg, Md.; 100 μl/well). Plates were incubated for 10 minutes and the absorbance was measured at 650 nm. Immunoreactivity was determined by comparing the concentrations of radiolabeled and unlabeled antibody where 50% of saturation binding was achieved.

7.2. Combination Therapy Using Paclitaxel and [⁹⁰Y-DOTA]776.1

Mice bearing established OVCAR-3 tumors were administered a single i.v. injection of [⁹⁰Y-DOTA]776.1 in 0.2 ml 0.9% sodium chloride. Groups of 8 mice received 50 μCi or 150 μCi [⁹⁰Y-DOTA]776.1. Additional groups were treated with 10 mg/kg paclitaxel, (which is lower than the MTD in nude mice (>30 mg/kg)), administered as a single dose or in weekly intervals of 10 mg/kg each for three weeks total (q3d7), or as a single dose in combination with radioimmunotherapy using yttrium-90 labeled antibodies at 50 μCi and 150 μCi doses. In groups receiving combination therapy, paclitaxel was administered either 24 hours prior to or 24 hours following administration of the radioimmunotherapy. Control groups consisted of mice injected with 0.9% sodium chloride alone. Control groups were also included that were injected with [⁹⁰Y-DOTA]MOPC-21 labeled to a similar specific activity as the [⁹⁰Y-DOTA]776.1 and evaluated at the same doses as the [⁹⁰Y-DOTA]776.1 groups. Tumors were measured two times per week. Mice were sacrificed when the tumor volume was greater than 10% of their body weight.

Statistical analysis of efficacy studies: Percent T/C, the ratio of mean tumor volume of treatment groups relative to the saline control group expressed as a percentage, was calculated for each group when the mean tumor volume of the saline control reached 1500 mm³ (see, e.g., Polin et al., Investig. New Drugs 15:99-108 (1997); Bissery et al., Cancer Res. 51:4845-4852 (1991)), found to be on day 40. Tumor volumes were compared by Mann-Whitney analysis using Prizm software (GraphPad Software, Inc.). Regression was defined as beginning when a tumor reached <50% of its starting volume and ending when the tumor reached >50% of its initial volume. Partial regression (PR) was defined as when a tumor decreased by 50% or more in volume for at least 7 days, and then later regrew. Complete regression (CR) was defined as when a tumor disappeared for at least 7 days. The combined effects of administering paclitaxel and ⁹⁰yttrium-labeled antibody were evaluated using the fractional product method (see, e.g., Yokoyama et al., Cancer Res. 60:2190-2196 (2000); Prewett et al., Clin. Cancer Res. 8:994-1003 (2002)).

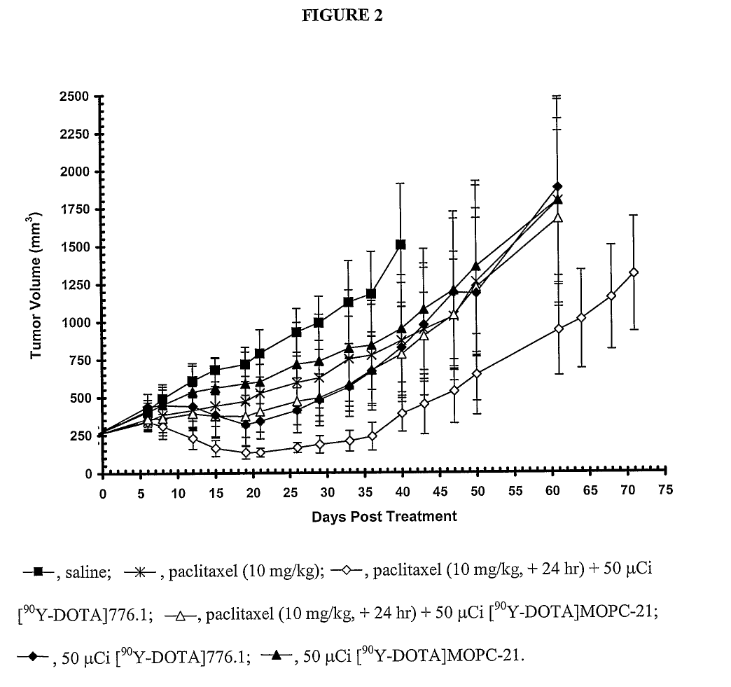

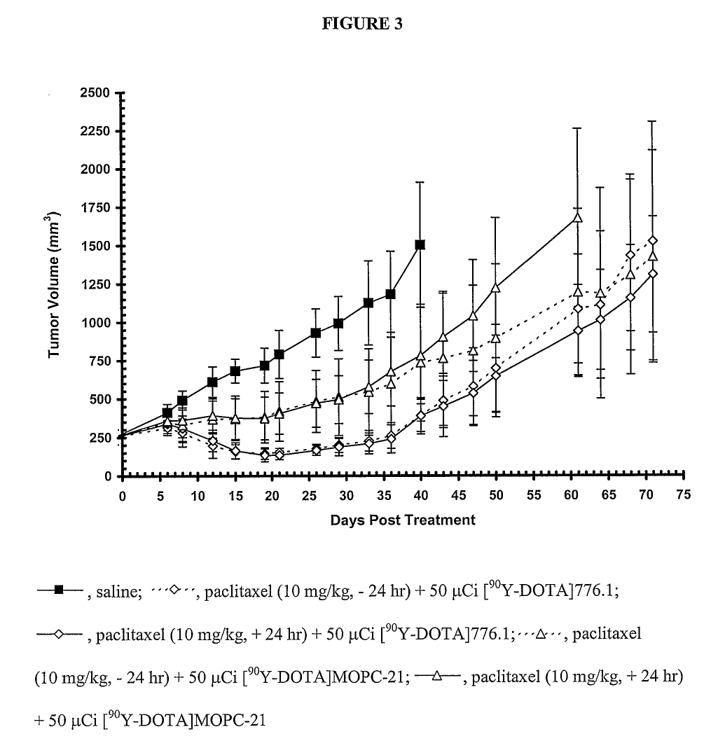

Effect of combination therapy on tumor growth: Treatment of mice with a combination of 50 μCi [⁹⁰Y-DOTA]776.1 and a single dose of 10 mg/kg paclitaxel either 24 hours prior to or 24 hours following radioimmunotherapy resulted in a significant enhancement of the reduction in tumor growth when compared with monotherapy with either paclitaxel (P=0.0003, 0.0148) or [⁹⁰Y-DOTA]776.1 alone (P=0.0019, 0.0104) administered at the same doses (FIG. 2). Partial regression up to 28 days was observed in 3/8 and 4/8 animals in those groups receiving a combination of low-dose radioimmnunotherapy and paclitaxel treatment, whereas no animals had observable regression of tumors in groups receiving low-dose radioimmunotherapy or paclitaxel treatment alone (Table 1). Combination therapy with low dose radioimmunotherapy and paclitaxel had a more significant effect on tumor growth than three weekly treatments with 10 mg/kg paclitaxel, as evidenced by a lower % T/C (26 versus 29), and a greater number of animals demonstrating regression of tumors (44% versus 10%). No difference was observed between combination therapy groups receiving paclitaxel 24 hours prior to or 24 hours following treatment with 50 μCi [⁹⁰Y-DOTA]776.1 (P=0.80) (FIG. 3).

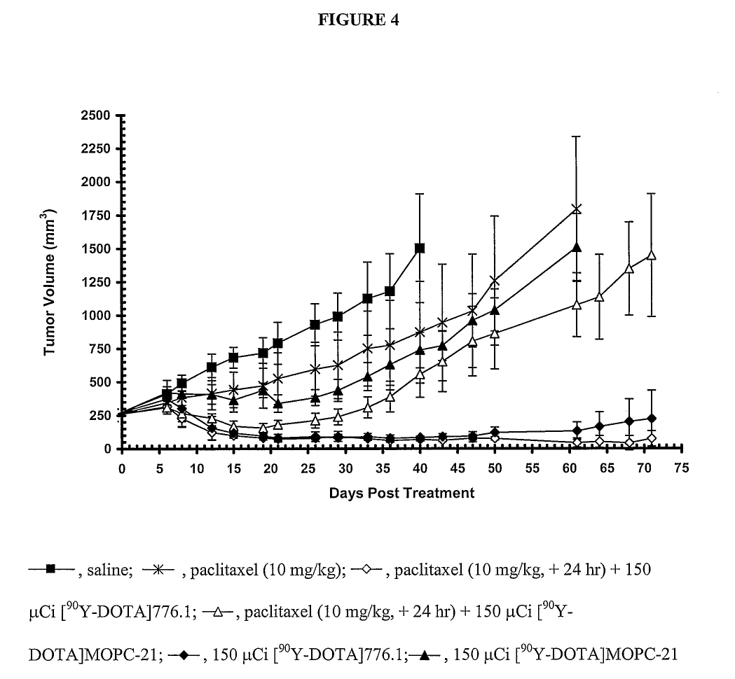

High-dose radioimmunotherapy using 150 μCi [⁹⁰Y-DOTA]776.1 was highly effective at slowing the growth of tumors; therefore it was difficult to compare the combined effects of radioimmunotherapy and chemotherapy at this dose (FIG. 4). However, a greater number of animals demonstrated complete regression of tumor in the combination therapy group (4/8 versus 1/8) and a statistically significant difference in the mean tumor volume at the end of the study was observed when compared with monotherapy using 150 μCi [⁹⁰Y-DOTA]776.1 alone (P=0.007). As controls, monotherapy and combination therapy were also performed using a ⁹⁰yttrium labeled non-specific antibody at similar doses. In all cases, treatment with [⁹⁰Y-DOTA]776.1 was much more effective than treatment with [⁹⁰Y-DOTA]MOPC-21.

TABLE 1
Effect of combined treatment of paclitaxel and [⁹⁰Y-DOTA]776.1 on the
growth of OVCAR-3 subcutaneous tumors

| | Tumor | Duration | | | | |
|---|---|---|---|---|---|---|
| Treatment | Regression | (days) | PR | CR | % T/C | FTV |
| Saline | — | — | — | — | 100 | — |
| Paclitaxel 10 mg/kg (day 0) | — | — | — | — | 58 | — |
| Paclitaxel 10 mg/kg (q3d7) | 1 | 14 | 1 | — | 29 | — |
| 50 μCi [⁹⁰Y-DOTA] 776.1 | — | — | — | — | 55 | — |
| Paclitaxel (−24) + 50 μCi [⁹⁰Y-DOTA] 776.1 | 3 | 2-28 | 1 | — | 26 | 1.23 |
| Paclitaxel (+24) + 50 μCi [⁹⁰Y-DOTA] 776.1 | 4 | 3-28 | 3 | — | 26 | 1.24 |
| 50 μCi [⁹⁰Y-DOTA] MOPC-21 | — | — | — | — | 63 | — |
| Paclitaxel (−24) + 50 μCi [⁹⁰Y-DOTA] MOPC-21 | — | — | — | — | 49 | 0.85 |
| Paclitaxel (+24) + 50 μCi [⁹⁰Y-DOTA] MOPC-21 | — | — | — | — | 52 | 0.80 |
| 150 μCi [⁹⁰Y-DOTA] 776.1 | 8 | 35-59 | 7 | 1 | 6 | — |
| Paclitaxel (+24) + 150 μCi [DOTA-⁹⁰Y] 776.1 | 8 | 59-63 | 4 | 4 | 5 | 0.71 |
| 150 μCi [⁹⁰Y-DOTA] MOPC-21 | — | — | — | — | 49 | — |
| Paclitaxel (+24) + 150 μCi [⁹⁰Y-DOTA] MOPC-21 | 2 | 7-18 | 2 | — | 37 | 0.80 |

Synergy between radioimmunotherapy and chemotherapy using paclitaxel was evaluated using the fractional product method (see, e.g., Yokoyama et al., Cancer Res. 60:2190-2196 (2000); Prewett et al., Clin. Cancer Res. 8:994-1003 (2002)). An FTV ratio of >1 indicates a synergistic effect, and a ratio of <1 indicates a less than additive effect. In groups receiving low dose radioimmunotherapy with [⁹⁰Y-DOTA]776.1 and a single dose of paclitaxel, a synergistic effect between the two treatments was observed, as evidenced by FTV values of 1.23 and 1.24 (Table 1). Due to the effectiveness of monotherapy using 150 μCi [⁹⁰Y-DOTA]776.1 alone, FTV ratios are below 1 in the group receiving high-dose radioimmunotherapy with [⁹⁰Y-DOTA]776.1 combined with paclitaxel treatment. For groups receiving combination therapy where the labeled antibody was [⁹⁰Y-DOTA]MOPC-21, no synergy was observed at either dose of [⁹⁰Y-DOTA]MOPC-21 tested. Thus, the synergistic effect observed between radioimmunotherapy and treatment with paclitaxel appears to be dependent upon the efficient targeting of the radiolabel by 776.1 to the tumor in this model system.

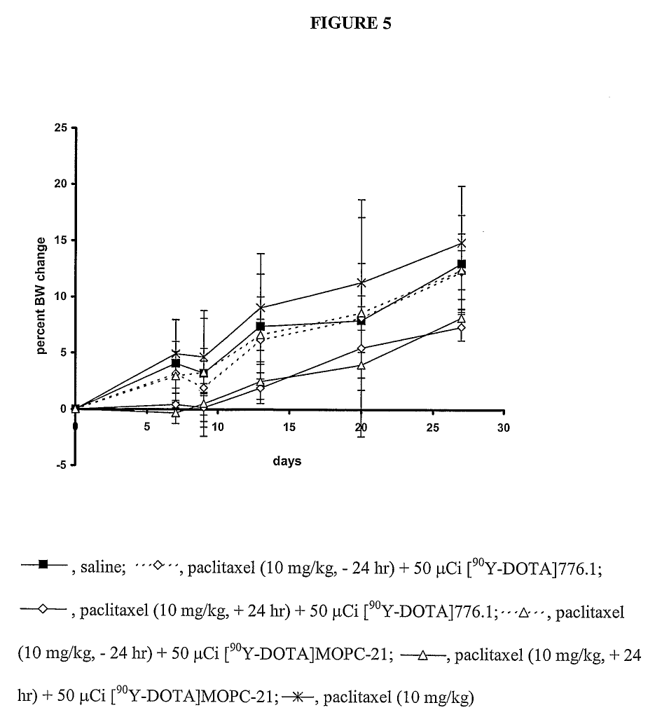

Toxicity: No dramatic toxicity, defined as a greater than 10% weight loss during the course of observation, was observed in any groups tested. Microcapillary damage was observed at early times following treatment in a subset of mice receiving high doses of radioimmunotherapy. This effect was transient, however, and all mice recovered. A significant delay in onset of weight gain was observed in mice that were treated with paclitaxel 24 hours following radioimmunotherapy treatment when compared with mice where treatment with paclitaxel was given prior to radioimmunotherapy (FIG. 5).

All publications, patents and patent applications mentioned in this specification are herein incorporated by reference into the specification to the same extent as if each individual publication, patent or patent application was specifically and individually indicated to be incorporated herein by reference. Citation or discussion of a reference herein shall not be construed as an admission that such is prior art to the present invention.

What is claimed is:

1. A method for managing a cell proliferative disorder in a subject in need thereof comprising administering to the subject
(a) a sensitizer, and
(b) an antibody, or an antigen-binding antibody fragment, that preferentially binds cell-associated CA 125 polypeptide relative to shed CA 125 polypeptide,
such that the cell proliferative disorder is managed.

2. The method of claim 1, wherein the cell proliferative disorder is cancer.

3. The method of claim 2, wherein the cancer is selected from group consisting of ovarian cancer, breast cancer, pancreatic cancer and hepatic cancer.

4. The method of claim 3, wherein the cancer is an ovarian cancer.

5. The method of claim 4, wherein the antibody, or the antigen-binding antibody fragment, is conjugated to a cytotoxic agent.

6. The method of claim 5, wherein the cytotoxic agent is a radionuclide.

7. The method of claim 5, wherein the cytotoxic agent is a non-radioactive cytotoxic agent.

8. The method of claim 1 or 4, wherein the sensitizer is paclitaxel.

9. The method of claim 1, wherein the subject is a human.

10. The method of claim 1, wherein the antibody or the antigen-binding antibody fragment is a monoclonal antibody (mAb), or an antigen-binding antibody fragment, that binds to a repeat region present within SEQ ID NO:1.

11. The method of claim 1, wherein the antibody or the antigen-binding antibody fragment is selected from a monoclonal antibody or fragment thereof which competes for binding to cell-associated CA 125 with a monoclonal antibody produced by hybridoma 4E7 (ATCC® Accession No. PTA-5109), or by hybridoma 7A11 (ATCC® Accession No. PTA-5110), or by hybridoma 7C6 (ATCC® Accession No. PTA-5111), or by hybridoma 7F10 (ATCC® Accession No. PTA-5112), or by hybridoma 7G10 (ATCC® Accession No. PTA-5245), or by hybridoma 7H1 (ATCC® Accession No. PTA-5114), or by hybridoma 8A1 (ATCC® Accession No. PTA-5115), or by hybridoma 8B5 (ATCC® Accession No. PTA-5116), or by hybridoma 8C3 (ATCC® Accession No. PTA-5246), or by hybridoma 8E3 (ATCC® Accession No. PTA-5118), or by hybridoma 8G9 (ATCC® Accession No. PTA-5119), or by hybridoma 15C9 (ATCC® Accession No. PTA-5106), or by hybridoma 16C7 (ATCC® Accession No. PTA-5107), or by hybridoma 16H9 (ATCC® Accession No. PTA-5108), or by hybridoma 117.1 (ATCC® Accession No. PTA-4567), or by hybridoma 325.1 (ATCC® Accession No. PTA-5120), or by hybridoma 368.1 (ATCC® Accession No. PTA-4568), or by hybridoma 446.1 (ATCC® Accession No. PTA-5549), or by hybridoma 501.1 (ATCC® Accession No. PTA-4569), or by hybridoma 621.1 (ATCC® Accession No. PTA-5121), or by hybridoma 633.1 (ATCC® Accession No. PTA-5122), or by hybridoma 654.1 (ATCC® Accession No. PTA-5247), or by hybridoma 725.1 (ATCC® Accession No. PTA-5124), or by hybridoma 776.1 (ATCC® Accession No. PTA-4570).

12. The method of claim 11, wherein the antibody or the antigen-binding antibody fragment is monoclonal antibody 776.1 produced by hybridoma 776.1 (ATCC® Accession No. PTA-4570), or an antigen-binding antibody fragment thereof.

13. The method of claim 10, wherein the sensitizer is paclitaxel and the mAb or antigen-binding mAb fragment is radiolabeled.

14. The method of claim 13, wherein the radiolabeled mAb or the radiolabeled antigen-binding mAb antibody fragment is radiolabeled with yttrium-90 (⁹⁰Y).

15. The method of claim 14, wherein the ⁹⁰Y-label is associated with the mAb or the antigen-binding mAb fragment by chelation to 1,4,7,10-tetraazacyclododecane-N,N′,N″,N′″-tetraacetic acid (DOTA) conjugated to the mAb or the antigen-binding mAb fragment.

16. The method of claim 1, 4 or 15, wherein the sensitizer is administered to the subject prior to administration to the subject of the antibody or the antigen-binding antibody fragment.

17. The method of claim 1, 4 or 15, wherein the sensitizer is administered to the subject after administration to the subject of the antibody or the antigen-binding antibody fragment.

18. The method of claim 1, 4 or 15, wherein the sensitizer is administered to the subject concurrently with administration to the subject of the antibody or the antigen-binding antibody fragment.

19. A method for managing an ovarian cancer in a human subject in need thereof comprising intravenously administering to the subject paclitaxel and a monoclonal antibody or antigen binding antibody fragment that preferentially binds cell-associated CA 125 polypeptide relative to shed CA 125 polypeptide, which antibody or antibody fragment binds a repeat region present within SEQ ID NO: 1, such that the ovarian cancer is managed.

20. The method of claim 19, wherein the antibody or antigen binding antibody fragment is conjugated to a cytotoxic agent.

21. The method of claim 20, wherein the cytotoxic agent is a radionuclide.

22. The method of claim 21, wherein the radionuclide is ⁹⁰yttrium.

23. The method of claim 20, wherein the cytotoxic agent is a non-radioactive cytotoxic agent.

24. A pharmaceutical composition comprising: (a) paclitaxel; (b) an antibody, or an antigen-binding antibody fragment, that preferentially binds cell-associated CA 125 polypeptide relative to shed CA 125 polypeptide; and (c) a pharmaceutically acceptable excipient, diluent, or carrier.

25. The pharmaceutical composition of claim 24, wherein the antibody or antigen binding antibody fragment is conjugated to a cytotoxic agent.

26. The pharmaceutical composition of claim 25, wherein the cytotoxic agent is a radionuclide.

27. The pharmaceutical composition of claim 26, wherein the radionuclide is ⁹⁰yttrium.

28. The pharmaceutical composition of claim 25, wherein the cytotoxic agent is a non-radioactive cytotoxic agent.

29. The pharmaceutical composition of claim 24, wherein the antibody is 776.1.
